(19) United States
(12) Patent Application Publication
(10) Pub. No.: US 2003/0176326 A1
(43) Pub. Date: Sep. 18, 2003

(54) Photosensitzers for photodynamic therapy of microbial infections

(75) Inventor: Nikolay E. Nifantiev, Moscow (RU)

Correspondence Address: BOLESH J. SKUTNIK PhD, JD, 515 Shaker Road, East Longmeadow, MA, 01028, US

(73) Assignee: CeramOptec Industries Inc.

(21) Appl. No.: 10/098,669

(22) Filed: Mar. 15, 2002

(51) Int. Cl. A61K 38/16; A61K 31/7052; C07K 14/47; C08B 37/00; A61K 31/739

(52) U.S. Cl. 514/8; 514/54; 514/42; 530/395; 536/17.4; 536/18.7; 536/53

(57) ABSTRACT

Molecular conjugates for the treatment and prevention of infectious diseases due to pathogenic microorganisms are provided. These conjugates comprise at least one photosensitizer coupled to a microorganism receptor (vector) that binds selectively to the surface of a microorganism, such as bacteria, viruses, mycoplasma, fungi, parasites and others. Complex aggregates formed by self-association of carbohydrate-photosensitizer conjugates or that are constructed on the base of a carrier form multiple interactions with the binding sites on the surface of the selected microorganism due to their polyligand carbohydrate surrounding. The property of the conjugates to bind selectively to sites on targeted microbes and to block these sites defines their ability to act as inhibitors of microbial cell adhesion and thereby provides an ability to prevent infectious disease. Methods for treatment and prevention of infectious diseases due to pathogenic microorganisms are also provided.

### BACKGROUND OF THE INVENTION

1. Field of the Invention

The present invention relates generally to photodynamic therapy, and particularly to molecular conjugates (photosensitizer conjugates) for the treatment and prevention of infectious diseases. A molecular conjugate of the present invention comprises at least one photosensitizer and at least one carbohydrate moiety.

2. Information Disclosure Statement

Photodynamic therapy (PDT) is one of the most promising new techniques being explored for use in a variety of medical applications and is known as a well-recognized treatment for the destruction of tumors (“Photodynamic therapy in hysterical perspective”, R. Bonnett, Rev. Contemp. Pharmacother. 10 (1999) pp. 1-17; “Potential applications of photodynamic therapy”, T. Okunara, H. Kato, Rev. Contemp. Pharmacother. 10 (1999) pp. 59-68; “Photodynamic therapeutics: basic principles and clinical applications”, W. M. Sharman, C. M. Allen, J. E. van Lier, DDT, 4 (1999) 507-517; “Pharmaceutical development and medical applications of porphyrin-type macrocycles”, T. D. Mody, J. Porphyrins Phtalocyanins, 4 (2000), pp.362-367; “Recent advances in photodynamic therapy”, R. K. Pandey, J. Porphyrins Phtalocyanins, 4 (2000), pp. 368-373; “Photodynamic therapy of skin cancers: sensitizers, clinical studies and future directives” F. S. De Rosa, M. V. L. B. Bentley, Pharmaceutical Research, 17 (2000) pp. 1447-1455; “Porphyrin-based photosensitizers for use in photodynamic therapy” E. D. Sternberg, D. Dolphin, C. Brueckner, Tetrahedron, 54 (1998) 4151-4202).

Another important application of PDT is the treatment of infectious diseases due to pathogenic microorganisms including dental, suppurative, respiratory, gastroenteric, genital and other infections.

A constant problem in the treatment of infectious disease is the lack of specificity of the agents used for the treatment of disease, which results in the patient gaining a new set of maladies from the therapy.

The use of PDT for the treatment of various types of disease is limited due to the inherent features of photosensitizers. These include their high cost, long retention in the host organism, substantial skin phototoxicity, background toxicity, low solubility in physiological solutions that reduces their usefulness for intravascular administration as it can provoke thromboembolic accidents, and low targeting effectiveness. These disadvantages lead to the administration of extremely high doses of a photosensitizer, which dramatically increase the possibility of accumulation of the photosensitizer in non-damaged tissues and the accompanying risk of affect to non-damaged sites.

One of the prospective approaches to increase the specificity of photosensitizers and the effectiveness of PDT is a conjugation of a photosensitizer with a ligand-vector, which specifically binds to receptors on the surface of a target cell. This approach is now used in the design of new generations of photosensitizers for the treatment of tumors (“Porphyrin-based photosensitizers for use in photodynamic therapy” E. D. Sternberg, D. Dolphin, C. Brueckner, Tetrahedron, 54 (1998) 4151-4202).

U.S. Pat. No. 5,466,681 describes a variety of conjugates useful for the treatment of infectious diseases due to pathogenic microorganisms. The conjugates comprise at least one agent coupled to a microorganism receptor—a carbohydrate vector, said vector is able to bind selectively to a microorganism. The agent is a penicillin antibiotic and said vector is an asialoganglioside or another carbohydrate chain. The conjugates are administered for the treatment of bacterial infections, particularly, caused by Streptococcus pneumoniae and by Helicobacter pylori.

A wide variety of natural and synthetic molecules recognized by target cells could be used as vectors. The use of oligopeptides and big protein molecules, including lectins, growth factors and especially antibodies to specific tumor cell antigens are known in the art. The '681 patent discloses a conjugate comprising at least one agent that is an anti-infective coupled to a microorganism receptor. Agents such as antibiotics, synthetic drugs and steroids are mentioned. Since photosensitizers do not themselves interact with microbes, they are not considered agents as described in the '681 patent and were not disclosed therein.

U.S. Pat. No. 5,696,000 discloses vectors selective for the pathogenic or opportunistic microorganisms, selected from the group consisting of Streptococcus agalactiae, Clostridium, Borrelia, Haemophilis parainfluenzae, Pseudomonas cepacia, Pseudomonas maltophilia, Neisseria meningitides, Coxiella and Shigella. Said vectors comprise substantially pure compounds, selected from the group consisting of Galβ1-4GlcNAcβ1-3Galβ1-4Glcβ1-1-X(R), Galβ1-3GlcNAcβ1-3Galβ1-4Glcβ1-1-X(R), GlcNAcβ1-3Galβ1-4Glcβ1-1-X(R), Galβ1-4GlcNAcβ1-3Galβ1-4Glc, Galβ1-3GlcNAcβ1-3Galβ1-4Glc, GlcNAcβ1-3Galβ1-4Glc, Galβ1-4GlcNAcβ1-3Gal, Galβ1-3GlcNAcβ1-3Gal, wherein X is sphingosine, hydroxylated sphingosine or saturated sphingosine and R is H or an N-acetyl fatty acid derivative of X. The patent discloses the use of these vectors for detecting and measuring pathogenic microorganisms. It also discloses the use of the receptors in a pharmaceutically acceptable carrier for treating or preventing illness or infection. Although the patent discloses the coupling of these receptors to antibiotics for targeting, it does not describe the coupling of these receptors with photosensitizers.

The attachment of use of carbohydrate moieties to improve the solubility of photosensitizers in physiological solutions is known. (“The synthesis of galactopyranosyl-substituted derivatives of pheophorbide”, A. A. Aksenova, Y. L. Sebyakin, A. F. Mironov, Russ. J. Bioorg. Chem., 26 (2000), pp.111-114; “Glycoconjugated Porphyrins. 3. Synthesis of flat amphiphilic mixed meso-(glycosylated aryl) arylporphyrins and mixed meso-(glycosylated aryl) alkylporphyrins bearing some mono- and disaccharide groups” D. Oulmi et al., J. Org. Chem., 60 (1995), pp.1554-1564).

Carbohydrate moieties are also known as vectors for molecular conjugates used for the treatment of tumors by PDT (“Synthesis and Biological Evaluation of Thioglycosylated Porphyrins for an Application in Photodynamic Therapy”, I. Sylvain, R. Zerrouki, R. Granet, Y. M. Huang, J.-F. Lagorce, M. Guilloton, J.-C. Blais, P. Krausz, Bioorg. Med. Chem. 10 (2002) 57-69).

The use of carbohydrate moieties as vectors for the treatment of infectious diseases by Photodynamic Therapy (PDT) has not been described. There is a need to develop molecular conjugates for the treatment of infectious diseases due to pathogenic organisms with a high targeting effectiveness and devoid of shortcomings mentioned above.

Among a wide variety of photosensitizers used for PDT, porphyrins and derivatives thereof are the ones of the most commonly used (“Pharmaceutical development and medical applications of porphyrin-type macrocycles”, T. D. Mody, J. Porphyrins Phtalocyanins, 4 (2000), pp.362-367; “Recent advances in photodynamic therapy”, R. K. Pandey, J. Porphyrins Phtalocyanins, 4 (2000), pp.368-373). Porphyrins and their derivatives have a high quantum yield for the formation of an exited triplet state. The difference between the energies of triplet state and ground-state oxygen makes them good energy donors to transfer the energy to the ground state to form singlet oxygen.

U.S. Pat. No. 5,217,715 discloses a carbohydrate receptor that is capable of specifically binding to many different species of bacteria. Subsequently, the receptor can be used to inhibit the attachment of pathogenic bacteria to diseased tissue in a preventive measure. The disclosed receptor is a purified carbohydrate compound that can be included in a composition having a pharmaceutically acceptable carrier. Although the carbohydrate receptor is disclosed in combination with an insoluble carrier or microtiter plate for the detection and removal of bacteria, the use of a carbohydrate receptor in combination with a pharmaceutical agent or photosensitizer is not disclosed.

U.S. Pat. No. 5,225,330 similarly discloses a diagnostic kit and diagnostic method utilizing carbohydrate receptors that are capable of adsorbing microorganisms. The carbohydrate receptors are immobilized on an insoluble substrate that is placed in contact with a sample to be tested for a particular microorganism. Although targeting and detection are described, the use of carbohydrate receptors to specifically target photosensitizers to microorganisms is not disclosed.

It is known in the prior art that several or many carbohydrate vectors may interact with several carbohydrate binding sites on the surface of microbial cells (See M. Mammen, S.-K. Choi and G. M. Whitesides, “Polyvalent interactions in biological systems: interactions for design and use of multivalent ligands and inhibitors”, Angew. Chem. Int. Ed., 37 (1998) 2754-2794; M. Monsigny, R. Mayer and A.-C. Roche, “Sugar-lectin interactions: sugar clusters, lectin multivalency and avidity”, Carbohydr. Lett., 4 (2000) 35-52). This is important because such oligo- and multi-site interactions are stronger than mono-site interactions, and the ability of molecular conjugates to act as inhibitors of microbial adhesion to host cells and thus prevent the initiation and further development of infectious diseases is thereby enhanced.

Carbohydrate-substituted porphyrins (conjugates of a photosensitizer moiety and a carbohydrate vector) and a number of other molecules, used as photosensitizers, under appropriate conditions are able to undergo self-assembling thus giving stable solutions (“Synthesis, self-assembling properties and incorporation of carbohydrate-substituted porphirins into cell membrane models”, C. Schell, H. K. Hombrecher, Chem. Eur. J., 5 (1999), pp. 587-598; “Self-assembly of disk-shaped molecules to coiled-coil aggregates with tunable helicity”, H. Engelkamp, S. Middelbeek, R. J. M. Nolte, Science, 284 (1999), pp. 785-788). It would be extremely useful to develop such self-assembled structures comprising a plurality of photosensitizers and a plurality of carbohydrate vectors. Such structures could selectively bind to a targeted microbial cell, and due to polyligand interactions, substantially block the sites on the targeted cell, thus preventing the targeted cell from binding to healthy host cells.

There remains a longstanding need for conjugates that can both prevent pathogenic infection by blocking specific binding sites and treat infectious diseases through high targeting effectiveness of conjugates that minimize adverse systemic effects.

### OBJECTIVES AND BRIEF SUMMARY OF THE INVENTION

An object of the present invention is to provide molecular conjugates for the treatment and prevention of infectious disease.

Another object of the present invention is to provide molecular conjugates for the treatment and prevention of infectious disease that selectively bind to several sites on the surface a targeted microbe.

Still another object of the present invention is to provide molecular conjugates for the treatment and prevention of infectious disease, comprising photosensitizers and carbohydrate vectors that target sites on a microbe surface.

Yet another object of the present invention is to provide a method for the treatment of infectious disease through the use of molecular conjugates of photosensitizers.

A further object of the present invention is to provide a method of prevention of infectious disease using molecular conjugates of photosensitizers as inhibitors of adhesion of a microbe to host cells.

Briefly stated, the present invention provides a variety of molecular conjugates for the treatment and prevention of infectious diseases due to pathogenic microorganisms. These conjugates comprise at least one photosensitizer coupled to a microorganism receptor (vector) that binds selectively to the surface of a microorganism, such as bacteria, viruses, mycoplasma, fungi, parasites and others. Complex aggregates formed by self-association of carbohydrate-photosensitizer conjugates or that are constructed on the base of a carrier form multiple interactions with the binding sites on the surface of the selected microorganism due to their polyligand carbohydrate surrounding. The property of the conjugates to bind selectively to sites on targeted microbes and to block these sites defines their ability to act as inhibitors of microbial cell adhesion and thereby provides an ability to prevent infectious disease. Methods for treatment and prevention of infectious diseases due to pathogenic microorganisms are also provided.

The above and other objects, features and advantages of the present invention will become apparent from the following description read in conjunction with the accompanying drawings.

### BRIEF DESCRIPTION OF FIGURES

FIG. 1A illustrates a direct connection between a photosensitizer and a carbohydrate vector.

FIG. 1B illustrates a spacer connection between a photosensitizer and carbohydrate vector.

FIG. 1C illustrates a bivectored conjugate.

FIG. 1D illustrates a trimeric carbohydrate cluster.

FIG. 1E illustrates a conjugate formed on a carrier.

FIG. 1F illustrates a dendrimeric conjugate having a photosensitizer nucleus.

FIG. 1G illustrates a dendrimeric conjugate having peripheral functionality.

FIG. 2 illustrates monovalent interactions between conjugates and binding sites on a targeted microbe.

FIG. 3 illustrates oligo- and polyvalent interactions between carbohydrate vectors and binding sites on a targeted microbe.

FIG. 4 illustrates a self-assembled complex bound to a plurality of binding sites on a targeted microbe.

### DETAILED DESCRIPTION OF PREFERRED EMBODIMENTS

Active conjugated compounds used in the present invention are generally photosensitizers attached to chemical devices acquiring biological specificity to target photosensitizers to defined kinds of microbes. A photosensitizer used in the present invention is generally any organic molecule suitable for PDT and capable of being directly, or via a stage of chemical activation, coupled to at least one carbohydrate vector. The list of photosensitizers includes natural products and their modified derivatives and synthetic molecules including but not limited to porphyrins, phtalocyanins, metallo derivatives thereof, dyes, synthetic photosensitizers and others. Particularly preferred photosensitizers include porphyrins and active derivatives thereof and synthetic photosensitizers.

Any carbohydrate molecular that is a natural receptor or is a part of the natural receptor participating in the microbial attachment to host cells can be used as carbohydrate vector. The list of carbohydrate vectors includes but is not limited to complete or partial carbohydrate chains of natural glycolipids, glycoproteins, oligo- and polysaccharides, proteoglycans, carbohydrate fragments of antibodies, selectively modified natural carbohydrate molecules, mimetics and analogs of natural carbohydrate molecules. In the case of vectors that are fragments of glycoproteins it is desired to use vectors containing mono- or oligopeptide fragment of protein chain together with a carbohydrate moiety.

Carbohydrate vectors are selected from the group consisting of but not limited to the following units:

| | |
|---|---|
| Galα | |
| Galβ | |
| Manα | |
| Manβ | |
| Neu5Acα | |
| Fucα | |
| GlcNAcβ | |
| GalNAc | |
| Galβ(1-4)Glcβ | (lactose) |
| Galβ(1-3)GlcNAcβ | (Le^{c}) |
| Galβ(1-4)GlcNAcβ | (LN) |
| Galα(1-4)Galβ | |
| Galα(1-4)Galβ(1-4)Glcβ | (Gb3, Pk) |
| GalNAcβ(1-3)Galα(1-4)Galβ(1-4)Glcβ | (Gb4) |
| Galβ(1-3)GalNAcβ(1-3)Galα(1-4)Galβ(1-4)Glcβ | (Gb5) |
| Galα(1-3)Galβ(1-4)Glcβ | |
| Galα(1-3)Galβ | |
| GlcNAcβ(1-3)Galβ | |
| GlcNAc(1-3)Galβ(1-4)Glcβ | |
| Galβ(1-3)GlcNAcβ(1-3)Galβ(1-4)Glcβ | (LNT) |
| Galβ(1-4)GlcNAcβ(1-3)Galβ(1-4)Glcβ | (LNnT) |
| Galβ(1-4)GlcNAcβ(1-3)Galβ(1-4)GlcNAcβ | |
| and larger oligo-lactosamines | |
| Neu5Acα(2-3)Galβ(1-4)Glcβ | (3′SL) |
| Neu5Acα(2-6)Galβ(1-4)Glcβ | (6′SL) |
| 3-O-sulfoGlcAβ(1-3)Galβ(1-4)GlcNAc | (HNK-1) |
| Neu5Acα(2-6)Galβ(1-4)GlcNAcβ(1-3)Galβ(1- | |
| 4)Glcβ | |
| [Neu5Acα(2-3)Galβ(1-4)GlcNAcβ(1- | |
| ]23,6Galβ(1-4)Glcβ | |
| Neu5Acα(2-3)Galβ(1-4)GlcNAcβ | (3′SLN) |
| Neu5Acα(2-3)Galβ(1-3)GlcNAcβ | (3′SLe^{c}) |
| Neu5Acα(2-6)Galβ(1-4)GlcNAcβ | (6′SLN) |
| Neu5Acα(2-3)Galβ(1-3)GlcNAcβ(1-3)Galβ(1- | |
| 4)Glcβ | (LSTa) |
| Neu5Acα(2-3)Galβ(1-4)GlcNAcβ(1-3)Galβ(1- | |
| 4)Glcβ | (3″′SLNnT) |
| Neu5Acα(2-3)Galβ(1-3)[Fucα(1-4)]GlcNAcβ(1- | |
| 3)Galβ(1-4)Glcβ | |
| Neu5Acα(2-3)Galβ(1-4)[Fucα(1-3)]GlcNAcβ(1- | |
| 3)Galβ(1-4)Glcβ | |
| Fucα(1-2)Galβ | H_{di} |
| Fucα(1-2)Galβ(1-3)GlcNAcβ | Le^{d} |
| Neu5Acα(2-3)Galβ(1-3)[Fucα(1-4)]GlcNAcβ | SiaLe^{a} |
| Galβ(1-3)[Fucα(1-4)]GlcNAcβ | Le^{a} |
| Fucα(1-2)Galβ(1-3)[Fucα(1-4)]GlcNAcβ | Le^{b} |
| Fucα(1-2)Galβ(1-4)GlcNAcβ | Hₜᵣᵢ |
| Neu5Acα(2-3)Galβ(1-4)[Fucα(1-3)]GlcNAcβ | SiaLe^{x} |
| Galβ(1-4)[Fucα(1-3)]GlcNAcβ | Le^{x} |
| Fucα(1-2)Galβ(1-4)[Fucα(1-3)]GlcNAcβ | Le^{y} |
| Galβ(1-3)GalNAcβ(1-4)Galβ(1-4)Glcβ | (asialo-GM1) |
| Galβ(1-3)GalNAcβ(1-4)[Neu5Acα(2-3)]Galβ(1- | |
| 4)Glcβ | (GM1) |
| GalNAcβ(1-4)Galβ(1-4)Glcβ | (asial-GM2) |
| GalNAcβ(1-4)[Neu5Acα(2-3)]Galβ(1-4)Glcβ | (GM2) |
| Fucα(1-2)Galβ(1-3)GalNAcβ(1-4)Galβ(1-4)Glcβ | (asial-FucGM1) |
| Fucα(1-2)Galβ(1-3)GalNAcβ(1-4)[Neu5Acα(2- | |
| 3)]Galβ(1-4)Glcβ | (FucGM1) |
| Manα(1-2)Man | |
| Manα(1-3)Man | |
| Manα(1-6)Man | |
| Manα(1-3)[Manα(1-6)]Man | |
| Manβ(1-2)Man | |
| Galβ(1-3)GalNAcα | |
| GlcNAcβ(1-3/6)GalNAcα | |
| Neu5Acα(2-8)Neu5Acα | |
| Fucα(1-2)[GalNAc═(1-3)]Galβ | |

and their fragments and substituted derivatives.

Other carbohydrate structures consisting of residues of neutral monosaccharides, aminosaccharides, sialic acid, or uronic acid may also be used as vectors. Vectors may also comprise sulfo groups and other substituents.

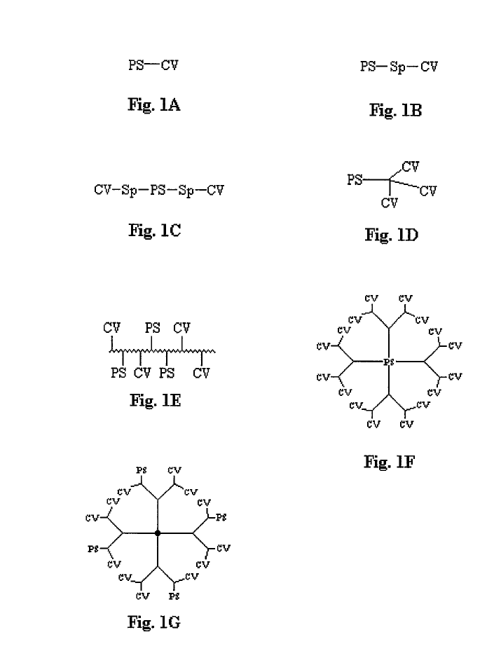

Referring to FIG. 1A through FIG. 1G, “PS” represents a photosensitizer residue or moiety, “CV” a carbohydrate vector, and “Sp” a spacer unit.

Preferred embodiments of the present invention include conjugates of photosensitizers and carbohydrate vectors that are connected directly (FIG. 1A) or via a spacer group (FIG. 1B). Vectored photosensitizers may comprise several carbohydrates attached to a photosensitizer, as in a bivectored conjugate (FIG. 1C). Photosensitizers may also be connected directly or via a spacer with a clustered carbohydrate construction containing several vector fragments, such as a trimeric carbohydrate cluster with three carbohydrate vector moieties (FIG. 1D).

In another preferred embodiment, carbohydrate vectors and photosensitizer moieties can be attached to a carrier directly or via a spacer (FIG. 1E). These conjugates can be designed based on complex moieties (matrixes) that can bind both a photosensitizer moiety and a carbohydrate vector directly or via a spacer. The aim of such a carrier is to provide a conjugate with several photosensitizers and several carbohydrate vectors, each or at least a majority of vectors capable of selective binding to the sites on a targeted microbe. Matrixes comprise but are not limited to, oligo- and polyamines, oligo- and polyacids, oligo- and polypeptides, dendrimers, polysaccharides and other carriers.

Other preferred embodiments of the present invention include two types of dendrimeric conjugates. A first type includes a photosensitizer unit that acts as a nucleus of a dendrimer (FIG. 1F). A second type of dendrimeric conjugate comprises photosensitizer and carbohydrate residues attached directly or via spacer groups to the peripheral parts of dendrimer branches (FIG. 1G).

Attachment of carbohydrate vectors is carried out by known preparative methods depending on the particular targeting structures. Conjugation should not affect the photodynamic properties of the photosensitizer or other important PDT characteristics. Conjugation can be carried out using inherent structural fragments of photosensitizers including but not limited to COOH, CHO, OH, C═O, NH, SH, halogen and other chemically active groups. Conjugation can also be accomplished by preliminary transfer of said photosensitizer to appropriately modified derivatives including but not limited to Br containing derivatives, or other suitable methods well known to those skilled in the art.

Preparation of conjugates of photosensitizers with carbohydrate vectors can be performed by different methods including but not limited to (a) direct glycosylation of photosensitizers, (b) coupling of photosensitizers with carbohydrate vectors via spacer groups, (c) coupling of carbohydrate moieties in an appropriate way and not via anomeric center, by immobilization of carbohydrate ligands and photosensitizers on (d) oligomer or (e) polymer carriers. Polymer carriers can be oligovalent matrixes, dendrimers, polyacrylic acid and derivatives thereof and other synthetic polymers, polysaccharides, polypeptides and others.

After conjugation with photosensitizers, carbohydrate chains can be elongated by chemical or enzymatic methods to generate most active carbohydrate structures to be used as vectors.

Conjugates of photosensitizers and carbohydrate vectors can alternately be designed by assembling carbohydrate vectors containing building blocks to form after the assembling of a photosensitizer moiety.

Carbohydrate ligand may fail to undergo direct binding to the photosensitizer residue so it may be necessary to use a spacer. A spacer between a photosensitizer and a carbohydrate vector should not change or affect recognition pathways of the carbohydrate vector and simultaneously should be of an appropriate size and structure so that the carbohydrate vector is not masked by photosensitizer residue. Any suitable spacers ordinarily known in the art can be used in the scope of the present invention.

It shall be noted that the majority of conjugates of the present invention are able to block the sites on the targeted microbe due to interactions of carbohydrate vectors with the carbohydrate binding sites on the microbe. These are the same binding sites that interact with carbohydrate molecules on the surface of host cells. Consequently, the molecular conjugates of the present invention act as inhibitors of adhesion of microbes to host cells and can be used for prevention of infectious diseases.

In yet another embodiment the conjugates of the present invention are self-assembling conjugates. Porphyrins, carbohydrated porphyrin conjugates and a number of other photosensitizing molecules are capable of self-assembling to form stable solutions. Complex moieties with polyligand carbohydrate surrounding result due to the process of self-assembling. The plurality of carbohydrate vectors, in addition to having other substantial advantages which will be discussed in detail below, is thought to improve the solubility of conjugates in physiological solutions.

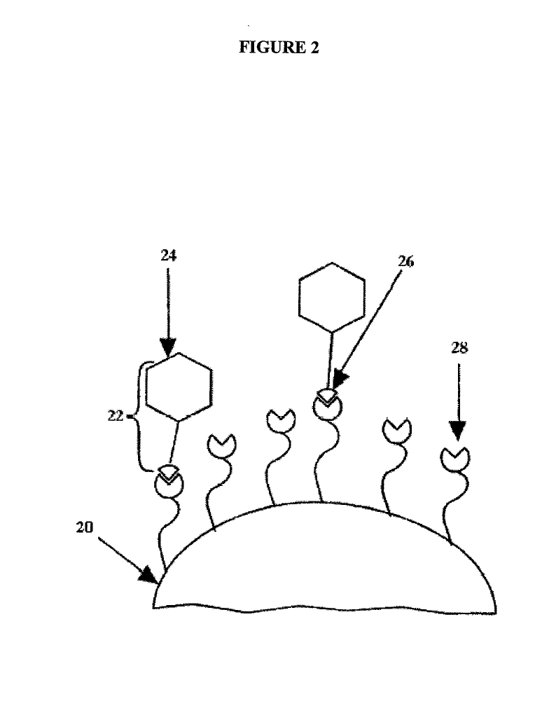

The targeting and anti-adhesion abilities of the carbohydrate vectors are illustrated in FIGS. 2, 3 and 4. Referring now to FIG. 2., conjugate 22 of the present invention comprising photosensitizer residue 24 and carbohydrate vector 26 attaches to binding sites 28 on targeted microbe 20 such as a bacteria, virus, micoplasma, fungi, parasite or other similar organism. The type of the carbohydrate vector is dependent on the nature of the targeted species.

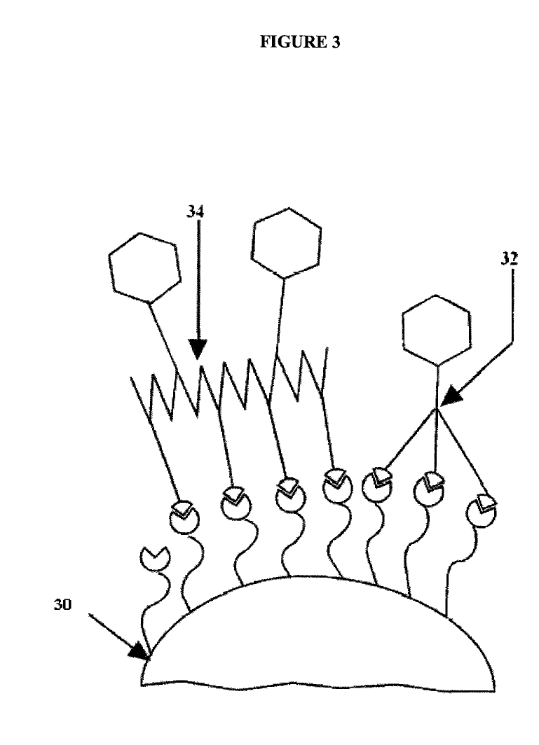

It is well known that many biological systems interact through multiple simultaneous molecular contacts or by simultaneous binding of multiple ligands on one biological entity to thus form polyvalent interactions. Referring to FIG. 3, targeted microbes 30 of the present invention may represent a plurality of carbohydrate binding sites ready for interaction with appropriate carbohydrate vectors. Molecular conjugates of photosensitizer moieties and several carbohydrate vectors are attached to suitable oligomeric carriers 32 and polymeric carriers 34 to produce oligomeric and polyvalent interactions. These types of interactions are stronger than monovalent interactions, and can more effectively prevent attachment of the microorganism to healthy cells of the host, preferably human, organism. Monovalent conjugates of the present invention are capable of acting as inhibitors to adhesion of microbes, but not as effectively.

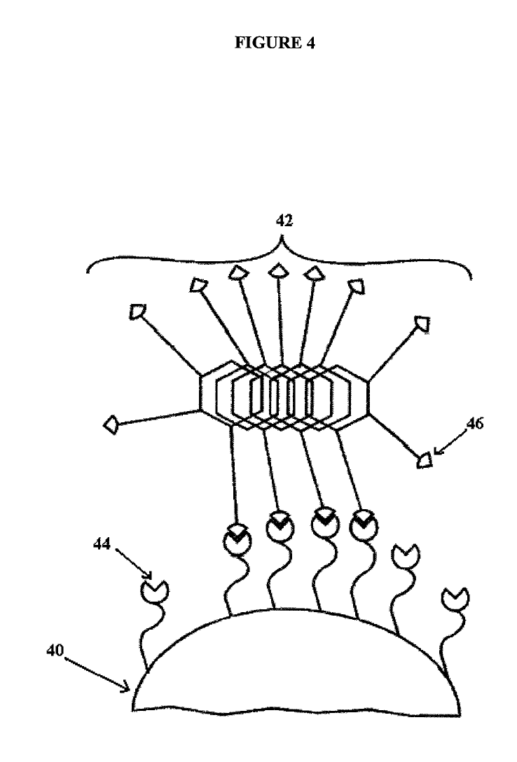

Self-assembled complexes 42 of the present invention are illustrated in FIG. 4. As a result of interactions between binding sites 44 on targeted microbe 40 and carbohydrate vectors 46 of conjugates of the present invention the binding sites on targeted microbe 40 that are responsible for the microbial adhesion to host cells become blocked. This important feature defines the ability of the above mentioned conjugates with one, several, and a plurality of carbohydrate vectors, self-assembled as well as non-assembled, to act as inhibitors for microbial cell adhesion and to be used for prevention of infectious disease caused by targeted microorganism.

Conjugates of the present invention are useful for the treatment of infectious diseases, caused by bacteria, viruses, fungi, micoplasma, parasites and other infectious agents. These include but are not limited to Pseudomonas aeruginosa, Pneumococcal pneumonia, enteroaggregative, uropathogenic and S-fimbriated Esherichia coli, Helicobacter pylori, Yersinia species, Clostridium difficile, Plasmodium species, Entamoeba histolitica, Streptococcus pneumonia, Streptococcus sanguis, Streptococcus sobrinus, Streptococcus mutants, Haemophilus influenzae, M. cattarhalis, Mycoplasma pneumoniae, Mycoplasma bovis, Candida albicans, Staphylococcus aureus, Porphyromonas gingivalis, Bacteroides forsythus, Influenza virus, HIV, Neisseria gonorroeae, Klebsiella pneumoniae, and Mycobacterium tuberculosis. The method of treatment of said diseases comprises administering to a host organism an effective amount of molecular conjugate of the present invention and irradiating the host organism with a wavelength that causes the photosensitizer to produce a cytotoxic effect.

Furthermore, conjugates of the present invention are used for prevention of the above mentioned diseases by administering to the host organism an effective amount of said conjugates.

It is expected that conjugates of the present invention will demonstrate an improved ability to be removed rapidly from the host organism due to their improved solubility in physiological solutions and will demonstrate reduced adsorption due to hydrophobic interactions. Furthermore, said conjugates are thought to possess low skin phototoxicity due to their improved ability to be removed from the host organism. High targeting effectiveness of said conjugates may lead to significant decrease in the doses of photosensitizers administered during therapy and also may lead to the reduction of side effects, especially background toxicity, and also may lead to improved selectivity of light irradiation.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A molecular conjugate, comprising
a) at least one photosensitizer moiety; and
b) at least one carbohydrate vector that selectively attaches to binding sites on a targeted microbe.

2. A molecular conjugate according to claim 1, wherein said photosensitizer is selected from a group consisting of porphyrins and active derivatives thereof, phtalocyanins, metallo derivatives thereof, dyes, and synthetic photosensitizers.

3. A molecular conjugate according to claim 2, wherein said photosensitizer is selected from a group consisting of porphyrins and active derivatives thereof.

4. A molecular conjugate according to claim 2, wherein said photosensitizer is a synthetic photosensitizer.

5. A molecular conjugate according to claim 1, wherein said carbohydrate vector is selected from a group consisting of complete or partial carbohydrate chains of natural glycolipids, glycoproteins, oligo- and polysaccharides, proteoglycans, carbohydrate fragments of antibodies, selectively modified natural carbohydrate molecules, mimetics and analogs of natural carbohydrate molecules.

6. A molecular conjugate, comprising
a) at least one photosensitizer moiety;
b) at least one carbohydrate vector that selectively attaches to binding sites on a targeted microbe; and
c) a carrier; wherein both said photosensitizer and said vector are attached to said carrier.

7. A molecular conjugate according to claim 6, wherein said photosensitizer is selected from a group, consisting of porphyrins and active derivatives thereof, phtalocyanins, metallo derivatives thereof, dyes and synthetic photosensitizers.

8. A molecular conjugate according to claim 6, wherein said carbohydrate vector is selected from a group consisting of complete or partial carbohydrate chains of natural glycolipids, glycoproteins, oligo- and polysaccharides, proteoglycans, carbohydrate fragments of antibodies, selectively modified natural carbohydrate molecules, mimetics and analogs of natural carbohydrate molecules.

9. A molecular conjugate according to claim 6, wherein said carrier is a complex moiety that attaches to both said photosensitizer moiety and said carbohydrate vector.

10. A molecular conjugate according to claim 9, wherein the method that said carrier attaches to said photosensitizer moiety and said carbohydrate vector is selected from a group consisting of direct connection, spacer connection, and a combination of direct and spacer connection.

11. A molecular conjugate of claim 6, wherein said carrier is selected from a group, consisting of oligo- and polyamines, oligo- and polyacids, oligo- and polypeptides, dendrimers, polysaccharides, polyacrylic acid and derivatives thereof.

12. A molecular conjugate according to claim 1, further comprising a plurality of photosensitizers associated with a plurality of carbohydrate vectors; said vectors selectively attach to binding sites on said targeted microbe; and whereby said photosensitizers together with said carbohydrate vectors undergo self-assembling to form a complex moiety.

13. A molecular conjugate according to claim 12, wherein said photosensitizers are selected from the group, consisting of porphyrins and active derivatives thereof, phtalocyanins, metallo derivatives thereof, dyes, and synthetic photosensitizers.

14. A molecular conjugate according to claim 12, wherein said vectors are selected from a group, consisting of complete or partial carbohydrate chains of natural glycolipids, glycoproteins, oligo- and polysaccharides, proteoglycans, carbohydrate fragments of antibodies, selectively modified natural carbohydrate molecules, mimetics and analogs of natural carbohydrate molecules.

15. A molecular conjugate according to claim 1, wherein said conjugate is an inhibitor of microbial cell adhesion.

16. A method for treatment of an infectious disease, comprising
a) administering to a host organism a pharmaceutically effective amount of the molecular conjugate of claim 1, and
b) irradiating said host organism with a wavelength that causes said photosensitizer to produce a cytotoxic effect.

17. A method for treatment of infectious disease of according to claim 16, wherein a cause of said disease is selected from a group consisting of bacteria, fungi, virus, micoplasma or parasites.

18. A method for prevention of an infectious disease, comprising administering to a host organism an effective amount of the molecular conjugate according to claim 15.

19. A method for prevention of infectious disease of claim 18, wherein a cause of said disease is selected from a group consisting of bacteria, fungi, virus, micoplasma or parasites.
